(19) United States
(12) Patent Application Publication
(10) Pub. No.: US 2012/0135529 A1
(43) Pub. Date: May 31, 2012

(54) Detection of Occult Blood in Feces or Urine

(76) Inventor: B. Eugene Guthery, Texarkana, TX (US)

(21) Appl. No.: 13/378,748

(22) PCT Filed: Jun. 15, 2010
(86) PCT No.: PCT/US10/38717

§ 371 Date: Feb. 10, 2012

Related U.S. Application Data
(60) Provisional application No. 61/218,246, filed on Jun. 18, 2009.

(51) Int. Cl. G01N 33/50; B01L 3/00

(52) U.S. Cl. 436/66; 422/430

(57) ABSTRACT

The present invention relates to an improved method, reagents and kit for the detection of fecal or urine occult blood. The reagents are either supplied as solutions in small vials or incorporated into a matrix that is treated, impregnated, or imprinted with the test reagents that are capable of undergoing a chromagenic reaction. The method elucidates false positives from dietary sources, oxidants in the toilet water supply and is specific for blood. A stabilized chromogenic solution is disclosed which contains phemolphthalin in a deoxygenated, basic solution. The chromagenic solution either in a liquid or matrix form is placed into the toilet water following urination or defecation. A few moments are allowed to occur for observation of the matrix turning from colorless to a hot-pink. A hot pink color is indicative of a false positive. If no color change occurs, an oxidizing solution is added to the toilet water. If a change from colorless to a hot-pink occurs in less than thirty seconds then this test is positive and specific for occult blood in this fecal and/or urine specimen. Typically, the oxidizing solution is hydrogen peroxide. Hemoglobin or a porphyrin solution can be added to a separate matrix or

### CROSS REFERENCE TO RELATED APPLICATIONS

This application claims the benefit of U.S. Provisional Application 61/218,246 filed Jun. 18, 2009.

### STATEMENT REGARDING FEDERALLY SPONSORED RESEARCH OR DEVELOPMENT

Not applicable.

### TECHNICAL FIELD OF INVENTION

The present invention relates to methods, reagents and kits for the detection of fecal or urine occult blood.

### BACKGROUND OF THE INVENTION

When there is blood in a fecal or urine specimen that can not be detected with the naked eye, the specimen is said to contain occult (hidden) blood. Occult gastrointestinal or urinary bleeding is often indicative of a variety of gastrointestinal, renal or bladder diseases. Examples of gastrointestinal diseases associated with occult gastrointestinal bleeding are adenomatous polyps and colon cancer. Examples of renal or bladder diseases that are associated with occult bleeding in the urine are bladder and kidney cancer.

A test for fecal occult blood was described by D. H. Greegor in 1969. See Cancer 19: 330-337 (1969). The Greegor test was based on the oxidation of guaiac to form a blue colored product in the presence of hydrogen peroxide and hemoglobin. U.S. Pat. No. 3,996,006 discloses a slide for use in testing fecal samples and suggests the use of guaiac or o-tolidine as possible reagents for fecal occult blood. In general, the test involves placing a fecal specimen on an absorbent paper that is coated with guaiac and adding a developer solution containing hydrogen peroxide. If hemoglobin is present in sufficient amounts in blood in the fecal specimen, the guaiac is oxidized, turning the paper blue. This test reportedly can detect (at least 50% of the time) a threshold level of occult blood resulting from bleeding by the patient of ten cc or more per day.

Purported improvements to the guaiac test have been reported in subsequent publications. European Publication Number 0 308 227 discloses addition of phenols to the developer solution to enhance the amount of colored product produced. Color enhancers are also described in U.S. Pat. Nos. 5,391,498 and 5,563,071.

U.S. Pat. No. 5,081,040 describes the use of 3,3′,5,5′-tetramethylbenzidine as a chromagen in a test for fecal occult blood. The chromagen and other components of the reagent are incorporated into a sheet of paper in a coated reaction area to form a test kit. In the method of use, a test kit is placed into a freshly-flushed toilet as a control. If no reaction occurs, a test kit is placed in the toilet water following defecation or urination. The paper should turn blue in the presence of sufficient hemoglobin to cause the color change reaction. The patent reports that 1.5 to 2.0 mg of hemoglobin per 100 cc of aqueous medium should be detectable.

Koch, et al. reported the use of phenolphthalin as an oxygen acceptor dye for use in assaying occult blood in fecal specimens. Canadian Journal of Medical Technology 35(3):13-14 (1973). However, the Koch reagent was reported to have a maximum shelf life of one week and the methodology required specimens to be collected and transported to the laboratory in a specific type of white cardboard container. The color change, if any, caused by occult blood in the specimen is directed to be observed on the white cardboard of the container.

Most recently, monoclonal antibody tests with a purported sensitivity of 50 micrograms hemoglobin/ml have been developed for testing of fecal occult blood. In such tests, water from the toilet in which a bowel movement has been deposited is sampled with a brush, placed on a slide and mailed to a testing laboratory for determination of results.

There is a continuing need to develop improved tests for the detection of occult blood with improved sensitivity, reduction of false positives, and which preferably are suitable for home testing by the patient.

### BRIEF DESCRIPTION OF THE DRAWINGS

FIG. 1 is a drawing depicting the chemical reaction which detects pseudo-perioxidase activity in a test specimen via a color reaction.

### DETAILED DESCRIPTION

Disclosed herein is a novel method, reagent and kit for the detection of occult blood in urine and feces. The method can detect a concentration of about 14 nanograms per ml or greater, which represents a significant increase in sensitivity over testing methods previously described in the art. The novel kit of the present invention is capable of detecting blood in a dilution of one to one hundred thousand (1:100,000). Increasing the sensitivity includes both detecting lower amounts of hemoglobin in the urine and/or fecal specimen and obtaining a clear distinction between positive and negative results.

In a preferred method for detecting occult blood, an assay is conducted in the toilet bowl where the fecal or urinary sample would ordinarily be deposited in the usual course of a person's normal routine. This is very convenient for the patient and therefore provides better compliance with testing. There is no need for the patient to try to take a sample of his or her excrement; thus this method avoids the step that may be either difficult or abhorrent to various individuals. Further, testing of a sample may not provide accurate results because the patient may inadvertently provide a sample that does not contain a sufficient amount of occult blood to be detected. It has been demonstrated that even though a patient is hemorrhaging the blood in the fecal specimen is not uniformly distributed throughout the fecal sample. If the patient takes a sample on a single occasion and testing of this sample is relied upon for determining if there is occult blood in the patient specimens, false negatives could easily result.

The preferred method is also advantageous for use in health care facilities. In-patient fecal or urine specimens can now be tested by nursing personnel in the patient's restroom. This technique allows patient care professionals to more accurately screen or monitor a fecal or urine specimen for occult blood and avoids the necessity of sampling, special slides or collection containers, sending the sample to the laboratory, and waiting for the results to come back from the laboratory before the health care professional can take further action to assist the patient.

In the preferred method of the invention, the entire bowel movement or urinary volume excreted is tested. In addition, it has been determined that when a fecal specimen containing blood on the surface of the feces comes in contact with toilet water in the toilet or commode, the blood separates from the fecal specimen and floats to the top of the commode water. This is an ideal circumstance for the novel invention to detect occult blood as it concentrates on top of the commode water.

The chromogenic composition used in the method of the invention is preferably stabilized through a method disclosed herein and thus has a shelf life suitable for home testing kits and health care facility on-site testing kits adapted for testing of occult blood. Examples of locations in which the kit may be desirably employed are locations remote from a testing laboratory such as homes, nursing homes, rehabilitation facilities, and hospital rooms. The sample can be tested at or near the time when the patient excretes waste material. Although the stabilized chromogenic composition can be used to detect occult blood in a patient sample, many advantages can be realized by using the reagent to detect occult blood as described for the preferred method.

A stabilized chromagenic composition is herein disclosed which is useful for detecting occult blood in fecal and/or urine specimens. The stabilized chromagenic composition comprises an aqueous basic solution of phenophthalin. The chromagenic composition remains highly stable over time by using a preferred method of making the composition. In this preferred method, deoxygenated water is utilized as the solvent and steps are taken in bottling or packaging which prevent oxidation. In a preferred embodiment, sodium or potassium hydroxide is included in the chromogenic composition to impart the basic character.

In another embodiment, a reducing agent is also included. A preferred reducing agent is zinc metal dust, but others can be used as long as compatible and non-reactive with the other ingredients other than to serve as a reducing agent.

Optionally, a hemoglobin solubilizing agent may be included in the chromagenic composition. The hemoglobin to be solubilized is the substance of interest for detection of occult blood in the specimen to be tested. A suitable hemoglobin solubilizing agent is an alcohol. Preferred alcohols are lower chain alcohols such as methanol, ethanol, or isopropyl alcohol. A hemoglobin solubilizing agent may be desirable if sampling methods are used that could bind the specimen to a collection instrument or there is a desire to free the hemoglobin from the specimen by solubilizing it. The chromagenic composition can be used in solution form or impregnated into an insoluble matrix.

In a method for testing for occult blood in a specimen, an appropriate control specimen is first tested with the chromagen. The control is selected so that it possesses all the attributes of the test specimen except the substance to be tested. For example, if a fecal specimen as deposited in a toilet containing water is the substance to be tested, an appropriate control specimen would be the toilet containing water prior to deposition of the fecal specimen. If an aliquot of a fecal specimen is to be tested after obtaining the same with a swab and placed in diluent, a control specimen would be a like swab placed in the same amount of diluent.

After ensuring that the control specimen does not cause a chromogenic reaction, the test specimen is then contacted with the chromagenic composition in an identical manner. A change in color to hot pink is indicative of a false positive if it occurs at this time alone prior to the addition of oxidizing compounds like hydrogen peroxide. If no change occurs, an oxidizer is then added. If a change in color to hot pink occurs after addition of the oxidizer, this is highly specific for occult blood. It is preferable to repeat the test on additional bowel movement specimens. In a preferred embodiment, the test is repeated on at least two additional consecutive bowel movements. If the first test is negative it is highly desirable to repeat the test on additional bowel movement specimens as gastrointestinal or renal diseases tend to hemorrhage intermittently.

If positive results are obtained, a health professional or patient will be able to investigate etiology of the occult bleeding and/or possible false positives. Because of the convenience of remote or home based testing that the invention makes possible, a more accurate assessment of the patient's health may be obtained than is possible with tests that must be conducted in a by a professional laboratory. This is because the test is conducted on a larger sample and can be repeated easily and quickly, thus enlarging the quantity of excreted material tested many fold.

Preferably, a patient to be tested will avoid certain foods, pharmaceuticals and antiseptics prior to testing with the chromagenic method disclosed herein. If not avoidable prior to testing, the test can still be conducted but the possible interfering cause noted. For example, foods, vitamins or supplements may cause a false positive result. Pharmaceuticals that may cause bleeding, such as corticosteroids, non-steroidal anti inflammatory agents (NSAIDS) such as aspirin, naproxen, and ibuprofen, anticoagulants, antimetabolites, and chemotherapeutic drugs should be noted as possible causative agents of a positive result. The consumption of alcohol prior to the test, especially if in excess, could cause a positive result. Rectal medications should be avoided if the feces are to be tested to avoid false positive results. In particular, preparations containing iodine should be avoided. The patient should abstain from foods containing peroxidases and excess Vitamin C in the diet for five days. Examples of foods to avoid are turnips, onions, radishes, and cantaloupe

If the specimen is to be tested in the toilet, use of toilet bowl cleaners, disinfectants or deodorizers could interfere with the test and should be avoided, particularly if they contain chlorine or bleach. In addition, the patient should remove chemical oxidants from the toilet water supply. In a preferred embodiment, a patient sample will be tested after deposition in a toilet in the normal manner. Table 1 summarizes the assay which will be detailed further herein.

TABLE 1

| Development of Hot Pink Color | Result | Interpretation |
|---|---|---|
| Color change occurs with | False- | Ignore results - The |
| chromogenic reagent alone with | Positive | patient must retest after |
| no added oxidants. | | following a peroxidase |
| | | restricted diet and/or |
| | | removal of oxidants |
| | | from the toilet water |
| | | supply. |
| No change with chromagenic | Positive | Occult Blood is present |
| reagent alone but color change | | in this specimen |
| occurs with chromagenic reagent | | |
| plus 3% Hydrogen Peroxide | | |
| No color change | Negative | No Occult Blood |

In one embodiment of the method, a toilet which is to be used for sample deposition should be flushed one or more times to ensure the water in which the sample will be deposited is generally clean. A test of the toilet water is then performed as a control. The chromagenic solution of the invention, a basic chromagenic deoxygenated solution containing phenophthalin, is added to the toilet. For example, an effective chromogenic solution for use in testing a fecal specimen deposited by a patient in a toilet bowl contains a concentration of the phenophthalin of about 2.0 grams per liter. One ounce (about 30 cc) is added to the toilet bowl, which generally will contain about 0.1 to 0.5 gallons or 0.1 to 2.0 liters of water.

In order to serve as an appropriate test site, the addition of the chromogenic solution should not result in a hot pink color when no specimen is present. If a hot pink color does develop, the toilet bowl must be cleaned of the substance causing the false positive. If this cannot be done, an alternative testing vehicle can be employed.

Once the toilet water is tested and deemed appropriate, clean water is again introduced and the used water removed as by flushing one or more times. The patient then defecates in the toilet in the normal manner and either the patient or an assisting person adds one ounce of the chromogenic solution to the toilet water. (The patient may wipe the anal area with toilet paper, but should put the used toilet paper aside). The water is then observed for 30 to 45 seconds. If a hot pink color develops, it is indicative of a false positive and testing should be stopped pending investigation of the cause for the false positive and elimination of said cause if possible.

If no hot pink color develops, an oxidizer solution is added to the toilet water containing the fecal deposit. A preferred oxidizer comprises 3% hydrogen peroxide. Any peroxide would work, for example urea peroxide. The important factor is to provide a peroxide that can be acted upon by the peroxidase-like activity of hemoglobin (also called pseudo-peroxidase activity). Hydrogen peroxide is commonly available in about 2.5 to 10% by weight for non-industrial applications. The weight percent of peroxide chosen should be of a sufficient amount to provide sufficient substrate per unit volume for the pseudo-peroxidase activity of the heme in the blood that could be present in a bowel movement to act upon so that the phenolphthalin (colorless) is oxidized to phenolphthalein (hot pink). One ounce (30 cc) of 3.0% hydrogen peroxide has been found sufficient for this purpose for an average bowel movement quantity. A patient with a 1 cm polyp may bleed approximately 1.2 cc a day, but the bleeding could be intermittent as previously discussed or excreted in several bowel movements. The preferred amounts specified (1 cc of a 2 gm/ml deoxygenated alkaline phenolphthalin solution and 1 cc of 3% hydrogen peroxide should be able to detect this type of bleeding, as well as any bleeding in excess of this threshold.

If the toilet water changes from clear to hot pink after addition of the oxidizer solution, the test is positive and specific for the presence of fecal occult blood. If no hot pink color occurs, this is indicative of the absence of occult blood.

Preferably, the test is repeated on two additional consecutive bowel movements. This will provide a more accurate picture of the patient's state of health because of the increase in sample size tested and the greater chance of detecting the intermittent bleeding that occurs naturally with bleeding lesions that cause occult blood to be present in patient excrement.

If there is a concern that occult blood may not be exposed to the reagent because it is trapped in the interior of the patient's stool, the stool can optionally be manually manipulated with a suitable object, such as a plastic stick, in the bowl in order to break it up and expose the interior to the reagent. Another method which will expose any occult blood to the reagent is for the patient to take a cathartic the evening before the test. An appropriate cathartic is magnesium citrate which can be taken according to package directions.

In another embodiment, the invention can be used in a bedpan. A patient's attendant can add approximately one quart (about one liter) of water to the deposited fecal sample in the bedpan. While the reaction will occur with direct addition to the fecal sample, detection is easier with the addition of water. Preferably, the bedpan will be tested prior to deposition of the sample to make sure there is not a false positive reading, rinsed out with the same supply of water tested for false positive readings, the sample deposited, then tested as described.

In an alternative embodiment, the chromogenic solution is affixed to a water insoluble matrix which can be used in the test method.

The reagent can be saturated into a water-insoluble matrix. Alternatively, the matrix is impregnated or imprinted with this reagent. Typically, the water-insoluble matrix is a sheet composed of fibers of cellulose.

The matrix is placed into the toilet water following urination or defecation. A few moments are allowed to occur for observation of the matrix turning from colorless to a hot-pink. If the matrix changes from colorless to hot-pink when placed on top of the toilet water this indicates a false positive and no further testing should be performed on this fecal and/or urine specimen unless and until the cause of the false positive is determined. When the patient is confident no color change occurs in the first few moments of observation when a water-insoluble matrix is placed into the toilet water an additional step should be performed by the patient. The patient should now add an oxidizing solution to the toilet water. If the matrix changes from colorless to a hot-pink in less than thirty seconds after the addition of the oxidizing solution, then this test is positive and specific for occult blood in this fecal and/or urine specimen. Typically the oxidizing solution is hydrogen peroxide, usually three percent.

In another embodiment, the oxidizer is also saturated onto the paper or insoluble matrix. In a method for using this embodiment, the insoluble matrix is added to the toilet and/or toilet containing the specimen. If a color change is observed, this is indicative of occult blood as long as false positives are ruled out.

In an alternative embodiment, the chromagenic reagent is supplied in a small vial or container. A heat resistant plastic, opaque vial is suitable. After defecation or urination, the lid of the vial is removed and the contents of the vial poured over a water-insoluble matrix like a cotton or cellulose filter. The filter is placed onto the top of the commode water following defecation and/or urination. If a color change occurs from colorless to hot-pink, this indicates a false positive (perhaps indicating that the patient ingested vegetables containing peroxidases or that there are chemical oxidants in the toilet water). After the patient obtains a reaction with no false-positive reactions the patient may test for occult blood in their fecal and/or urine specimens.

In the absence of a spontaneous color change, the patient should add an oxidizing solution to the toilet water. If the cellulose filter changes from colorless to a hot-pink in less than thirty seconds after adding an oxidizing solution, then this test is positive and specific for occult blood in this fecal and/or urine specimen.

Care should be taken in making the chromagenic solution to use

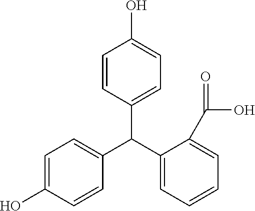

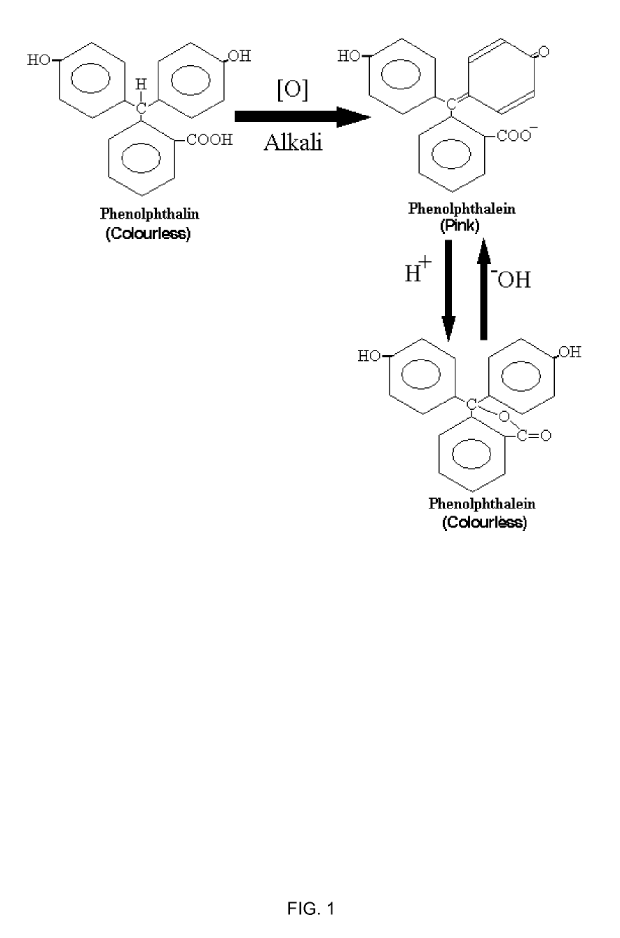

phenophthalin rather than phenolphthalein. The structure of phenolphthalin is provided above. It is available from Sigma-Aldrich, Catalogue Number P8903, TCI America Catalogue Number P0095 and may be available from other chemical suppliers. The formula for phenolphthalin is 2-(Bis[4-hydroxyphenyl]methyl)benzoic acid. The reaction which occurs is provided in FIG. 1.

The test is indicative of heme in the blood because hydrogen peroxide (H₂O₂) will react with any heme that might be present in the sample because of the pseudo-peroxidase activity of the heme. The pseudo-peroxidase activity of hemoglobin decomposes peroxide into water (H₂O) and a free oxygen radical (O) The free oxygen radical is attracted to the phenolphthalin color-indicator as prepared. The oxygen oxidizes the phenophthalin, and this oxidation causes it to turn pink. The color change is observable under normal room lighting conditions. The sodium salt of phenolphthalin is colorless in deoxygenated alkaline solution, and is readily oxidized by minute quantities of blood in the presence of hydrogen peroxide to phenolphthalein which gives a deep hot pink color in alkaline solution.

Phenophthalin solutions have been used in the field of forensics to detect blood at crime scenes. Koch et al reported a use of a phenophthalin alcohol-based preparation of limited stability for fecal occult blood screening, but only under laboratory conditions. However, such phenophthalin preparations have not been suitable for use in a kit for occult blood detection due to the short stability and shelf life. Moreover, making a stable reagent has been reported to be very difficult.

It has now been found that the stability, and therefore the shelf-life can be extended substantially by the herein disclosed preparation technique. To prepare a chromogenic reagent which will remain stable over time, it is necessary to deoxygenate the water used as a solvent. This can be done by boiling the water to be used exhaustively. Other methods to deoxygenate water are known in the art, such as low pressure deaeration or bubbling with nitrogen, argon or helium gas. In order to extend the shelf-life, the phenophthalin must be prepared in such a fashion that there is no residual oxygen in the solution.

An alkali, or donor of -OH ions, is then added to the deoxygenated water. Preferred alkalis are sodium hydroxide or potassium hydroxide. The amount that should be added is approximately 0.25 moles per liter. This raises the pH of the solution to approximately 8 or more. It is imperative that the developing solution contains enough hydroxide ion to raise the commode water to a pH above 8. The largest commode is usually 1.8 liters and 20 grams of sodium hydroxide per liter is capable of raising the pH above 8.0 when only 30 cc is added to the commode water.

Phenophthalin can be added to the hot, basic solution at an amount of about 2.0 grams per liter. One ounce (30 cc) of a solution containing 20 grams of sodium or potassium hydroxide and 60 micrograms of phenophthalin are added to the commode. The largest commode contains 1.8 liters of water. The final concentration of phenophthalin in a commode thus is preferably at least about 33.3 micrograms per liter. The concentration of the reagent and or the amount of the reagent can be adjusted to achieve this approximate concentration in the commode water.

A titration was performed with reagents containing1, 2 or 5 grams of phenolphthalin per liter and the reaction was found to reach maximum intensity change using about one ounce (30 cc) of a reagent having approximately 2 grams per liter. Increasing the concentration can be done, but will not increase the intensity to a great degree.

The chromagenic composition and method of the invention increase the hemoglobin detection limits substantially. This amount of phenophthalin is capable of detecting 14 nanograms of hemoglobin/ml in a 1.8 liter Erlenmeyer flask when combined with one ounce of 3 percent hydrogen peroxide. Excess hydrogen peroxide does not quench the reaction.

The boiling technique has advantages in packaging the reagent. The water is boiled prior to adding the phenophthalin and sodium hydroxide. The phenophthalin solution should be filled to the top of the bottle while hot to avoid the presence of any air. The lid is placed on the bottle while the solution is still hot. Preferably, an auxiliary seal composed of oxygen-impermeable material, such as a foil or film is used to seal the bottle, then another lid is placed over said seal.

An alternate method is to fill the bottle with room temperature solutions and add a gas, such as liquid nitrogen, to remove any oxygen from the solution. The seal and lid is preferably employed.

A reducing agent may be optionally added to the chromagenic solution. One reducing agent that may be used is zinc metal dust, but others may be used as long as they will not interfere with the desired test reaction. This will help prevent the phenophthalin from turning a pink color in the reagent bottle. If the reagent is prepared by using boiled deoxygenated water to which the hydroxide ion donor is added while hot, followed by the phenophthalin and the solution bottled while hot, the need for a reducing agent is diminished.

Alcohol may be optionally added to the chromogenic solution or supplied as a separate component of a kit. The purpose of alcohol is to assist in solubilizing hemoglobin so that it is freed up to react with the chromogenic reagent. Any alcohol may be used, but most preferably isopropyl, ethanol or methanol will be used.

The kit may also include a positive control. A solution of hemoglobin is made to contain 2 grams of hemoglobin per one hundred ml of normal solution. One or two drops of this hemoglobin solution can be placed in the middle of a paper filter saturated with the novel chromagen solution. Porphyrin solutions may also be used as a positive control.

### EXAMPLE 1

### Making of Chromagenic Test Paper

Sodium hydroxide (10 grams) sodium phenophthalin (2.0 grams), and zinc metal dust (1.0 gram) was mixed in deionized water and brought up to one liter. The solution was boiled for one hour to deplete the solution of oxygen. When the solution turned colorless this was taken as an indication that most of the oxygen had been removed. Isopropyl alcohol (400 cc) and additional de-oxygenated water was added to return the total volume to one liter.

The above solution was poured over paper filters to saturate each filter with the chromagen solution in an oxygen depleted environment. In this example, one or two paper filters were placed into a hermetically sealed envelope under a stream of nitrogen. The seal on the hermetically sealed envelope was closed to prevent exposure to the ambient air or light.

I claim:

1. A test kit for the detection of occult blood in fecal and/or urine specimens comprising:
a. a stable chromogenic reagent comprising phenophthalin in a deoxygenated basic aqueous solution; and
b. an oxidizing reagent comprising an oxygen donor capable of oxidizing phenophthalin to phenolphthalein and a change of color from colorless to pink.

2. The test kit of claim 1, further comprising an insoluble matrix to which said chromogenic reagent is affixed.

3. The test kit of claim 2, wherein said insoluble matrix comprises fibers of cellulose.

4. The test kit of claim 2, wherein said insoluble matrix to which said chromogenic reagent is affixed is packaged in a sealed pouch which is capable of preventing oxidation of said reagent during storage.

5. The test kit of claim 1, wherein said chromogenic reagent comprises deoxygenated water, a hydroxyl donor and phenophthalin in a deoxygenated vial.

6. The test kit of claim 1, wherein said chromogenic reagent comprises deoxygenated water, a hydroxyl donor selected from sodium and potassium hydroxide, and phenophthalin in a deoxygenated vial.

7. The test kit of claim 1, wherein said chromogenic reagent comprises deoxygenated water, a hydroxyl donor and a reducing agent.

8. The test kit according to claim 1, wherein the oxidizing agent comprises hydrogen peroxide.

9. The test kit of claim 1, wherein said chromogenic reagent further comprises alcohol in a sufficient amount to dissolve hemoglobin in the specimen to be tested.

10. The test kit of claim 1, further comprising a positive control.

11. The test kit of claim 10, wherein said positive control is selected from the group consisting of hemoglobin solutions and porphyrin solutions.

12. A method for testing a specimen for blood, comprising the steps of providing a chromagenic reagent comprising phenophthalin to a specimen deposited in a toilet bowl and observing for a visually observable change of color only after adding an oxidizing solution.

13. The method of claim 12, further comprising the step of observing for false positives by observing for a change in color after addition of a chromagenic reagent but before adding said oxidizing solution.

14. The method of claim 12, further comprising the step of testing the toilet bowl with chromagenic reagent prior to deposition of the specimen in the toilet bowl to ensure that the test environment does not cause false positive readings.

15. The method of claim 12, wherein said chromagenic agent is a deoxygenated basic solution of phenolphthalin.

16. The method of claim 15, wherein said phenolphthalin is in a concentration of about 1 to about 5 g/liter in said reagent.

17. The method of claim 15, wherein said phenolphthalin is in a concentration of about 2 g/liter in said reagent.

18. A method for making a stable phenophthalin reagent, comprising the steps of:
a. deoxygenating water by boiling;
b. adding a hydroxyl donor to said water in a sufficient amount to form a deoxygenated basic solution, one ounce of which is capable of changing the pH of from about 1.5 to 10 gallons of water to pH 8 or above;
c. dissolving phenophthalin in said deoxygenated basic solution to form a phenophthalin reagent;
d. bottling said phenophthalin reagent under conditions which prevent oxidation.

19. The method of claim 18, wherein said conditions which prevent oxidation comprise bottling while still hot and sealing.

20. The method of claim 18, wherein said conditions which prevent oxidation comprise bottling said phenophthalin reagent under inert gas and sealing to prevent oxidation.

21. The method of claim 19, wherein said seal is gas impermeable.

22. The method of claim 20, wherein said seal is gas impermeable.

23. A stable deoxygenated phenolphthalin reagent, comprising from 1.0 to 5.0 grams/liter of phenolphthalin in a basic solution.

24. The reagent of claim 23, wherein said phenolphthalin is about 2.0 grams/liter.

25. The reagent of claim 23, comprising about 0.5 M sodium hydroxide.

26. The reagent of claim 23, further comprising an alcohol component.

27. The reagent of claim 24, further comprising a reducing agent.

28. The reagent of claim 27, wherein said reducing agent is zinc.

29. The reagent of claim 23, further comprising an insoluble matrix to which said reagent is affixed
